(19) United States
(12) Patent Application Publication
(10) Pub. No.: US 2007/0231406 A1
(43) Pub. Date: Oct. 4, 2007

(54) Use of gallium to treat biofilm-associated infections

(76) Inventors: Louis R. Bucalo, Miami, FL (US); Susan Schwendner, Berkeley, CA (US); Uwe F. Wirtz, Oakland, CA (US); Sunil Sreedharan, South San Francisco, CA (US)

Correspondence Address: MORRISON & FOERSTER LLP, 755 PAGE MILL RD, PALO ALTO, CA, 94304-1018, US

(21) Appl. No.: 11/700,289

(22) Filed: Jan. 30, 2007

Related U.S. Application Data
(60) Provisional application No. 60/763,676, filed on Jan. 30, 2006.
(60) Provisional application No. 60/801,082, filed on May 16, 2006.

(51) Int. Cl. A61K 33/24

(52) U.S. Cl. 424/617

(57) ABSTRACT

The invention provides methods, compositions, and kits for treatment or prevention of biofilm-associated infections in an individual. Methods of the invention include administration of a gallium-containing composition for treatment of an established biofilm or prevention of formation of a biofilm. Some methods include administration of a gallium-containing composition in conjunction with an antibiotic substance. Some methods include treatment or prevention of an orally-associated biofilm with a gallium-containing composition in the form of a dentrifice, mouthwash, or chewing gum composition.

### CROSS-REFERENCE TO RELATED APPLICATIONS

This application claims the benefit of U.S. Provisional Application No. 60/763,676, filed on Jan. 30, 2006, and U.S. Provisional Application No. 60/801,082, filed on May 16, 2006, the disclosures of both of which are incorporated herein in their entireties.

### FIELD OF THE INVENTION

The invention relates to the use of gallium-containing compositions for treatment of an existing biofilm or prevention of the formation of a biofilm in an individual.

### BACKGROUND

Bacterial biofilms are colonies of bacteria encapsulated by an extracellular matrix.

The bacteria encapsulated in biofilms are often relatively impervious to detergents and antibiotics. Antibiotic resistance of bacteria in biofilms has been extensively documented and bacterial biofilms play a role in a number of disease settings, including the exacerbation of cystic fibrosis, chronic urinary tract infections, chronic sinus infections, infections due to medical devices such as catheters and ventilators, and dental plaque. (See, e.g., Costerton et al. (1999) Science 284(5418)1318-22.)

The chemical element iron is required for biofilm formation and maintenance, and pathogenic bacteria have evolved specialized mechanisms for extracting iron from the host. For example, the opportunistic pathogen Pseudomonas aeruginosa in cystic fibrosis and urinary tract infections, expresses two siderophores, pyocydin and pyoverdin, to capture extracellular iron from the host environment. Pseudomonas aeruginosa biofilm formation has been shown to be inhibited by iron sequestration in the presence of 20 μg/ml lactoferrin, a key iron-binding protein expressed in the host's mucosal secretions (Singh et al. (2002) Nature 417(6888):552-55). As a consequence, these bacteria were sensitive to macrolide antibiotics and other aminoglycosides such as tobramycin. Replenishing the media with iron resulted in a significant bacterial regrowth and formation of antimicrobial-resistant bacterial biofilms. Time-lapse microscopy demonstrated that the sequestration of iron by lactoferrin induces P. aeruginosa to roam across a surface instead of forming microcolonies and aggregating into biofilms. Iron-rich conditions stimulate a phenotype whereby bacteria form cell clusters and thereafter biofilms.

The principle pathogens of chronic urinary tract infections (UTI) are Gram-negative rods such as Escherichia coli, Proteus spp., and Klebsiella pneumoniae, all three of which have been shown to form biofilms. Although the number of available antibiotics to treat UTI has increased, so has the prevalence of resistant pathogens. Resistance in UTI pathogens is due to various factors, one of which is the formation of bacterial biofilms.

Bacterial biofilms are associated with catheter-associated UTIs, struvite calculogenesis, and chronic prostatitis, as well as other common UTI scenarios. Biofilm associated bacterial infections are often nosocomial in nature and flare up acutely in UTIs, lung infections in intensive care units, skin infections in burn victims, and septicemia associated with neutropenic cancer. Coagulase-negative Staphylococci, Enterococcus spp., Klebsiella pneumoniae, and Pseudomonas aeruginosa are commonly associated with biofilms on urinary catheters.

Antibiotic resistance of bacteria embedded in biofilms adhering to urinary catheters is well documented (Costerton et al., supra). The increased resistance to antibiotic therapy in such biofilms may be secondary to poor antibiotic penetration into the biofilm matrix itself or to decreased metabolic activity within the biofilm. Individual bacteria dispersed from antibiotic-resistant biofilms regain sensitivity to low levels of antibiotics once they lose the protection of the biofilm environment.

There is a need in the art for improved methods for treating or preventing biofilm formation.

### BRIEF SUMMARY OF THE INVENTION

The invention provides methods, compositions, and kits for treating a biofilm in an individual in need thereof.

In one aspect, the invention provides a method for treating a biofilm in an individual in need thereof, comprising administering a therapeutically effective amount of a gallium-containing composition to the individual. Treatment includes prophylaxis, therapy, or cure. In some embodiments, the method comprises prevention of formation of a biofilm, comprising administering a prophylactically effective amount of a gallium-containing composition to an individual. In one embodiment, the method comprises inhibition or prevention of spread of a biofilm-associated infection to another site in the individual. In another embodiment, the method comprises breaking the extracellular biofilm matrix and thus enabling the host's immune system to clear the infection.

In various embodiments, the biofilm is present in the bladder, the kidney, the heart, the middle ear, the sinuses, the skin, the lung, a joint, subcutaneous tissue, soft tissue, vascular tissue, and/or the eye. In one embodiment, the method comprises treatment of a biofilm associated with a urinary tract infection. In another embodiment, the biofilm is associated with chronic bacterial vaginosis. In another embodiment, the biofilm is associated with bacterial keratitis. In one embodiment, the biofilm is associate with prostatitis. In one embodiment, the biofilm is in the lung of an individual wherein the individual does not have cystic fibrosis. In one embodiment, the biofilm is in the lung of an individual wherein the biofilm does not comprise Pseudomonas aeruginosa. In one embodiment, the biofilm is on the skin of an individual wherein the skin does not comprise a burn wound.

In some embodiments, the biofilm comprises at least one bacterial species. The bacterial species may be a Gram-positive or a Gram-negative species. Gram-positive species include, but are not limited to, Bacillus, Corynebacteria, Clostridium, Enterococcus, Listeria, Staphylococcus, or Streptococcus. Gram-negative species include, but are not limited to, Pseudomonas aeruginosa, Branhamella, Campylobacteria, Escherichia coli, Enterobacteria, Pasteurella, Proteus, Klebsiella, Neisseria, Salmonella, Shigella, or Serratia.

In some embodiments, the method comprises administering at least one antibiotic substance in combination with the gallium-containing composition. Administration of the at least one antibiotic substance may be simultaneous or sequential with respect to administration of the gallium-containing composition. In some embodiments, the antibiotic substance works synergistically with the gallium-containing composition to treat the biofilm. In some embodiments, the antibiotic substance works additively with the gallium-containing composition to treat the biofilm. Antibiotic substances that may be used in accordance with methods of the invention include, but are not limited to, ciproflaxin, ampicillin, azithromycin, cephalosporin, doxycycline, fusidic acid, gentamycin, linezolid, levofloxacin, norloxacin, foloxacin, rifampin, tetracycline, tobramycin, vancomycin, amikacin, deftazidime, cefepime, trimethoprim/sulfamethoxazole, piperacillin/tazobactam, aztreanam, meropenem, colistin, and chloramphenicol. Classes of antibiotic substances that may be used in accordance with the methods of the invention include, but are not limited to, aminoglycosides, carbacephem, carbapenems, first generation cephalosporins, second generatin cephalosporins, third generation cephalosporins, fourth generation cephalosporins, glycopeptides, macrolides, monobactam, penicillins, polypeptides, quinolones, sulfonamides, tetracyclines, lincosamides, and oxazolidinones.

In some embodiments, the gallium-containing composition comprises a coordination complex in the form of a neutral 3:1 (hydroxypyrone:gallium) complex in which each hydroxypyrone molecule is either unsubstituted or substituted with one, two, or three C₁-C₆ alkyl substituents. In some embodiments, each hydroxypyrone molecule is selected from the group consisting of 3-hydroxy-4-pyrone, 3-hyroxy-2-methyl-4-pyrone, 3-hydroxy-2-ethyl-4-pyrone, and 3-hydroxy-6-methyl-4-pyrone. In one embodiment, each hydroxypyrone molecule is 3-hydroxy-2-methyl-4-pyrone.

In some embodiments, the gallium-containing composition is administered parenterally. In some embodiments, the gallium-containing composition is administered orally. In some embodiments, the gallium-containing composition is administered locally or topically.

In another aspect, the invention provides a method for treating a biofilm in an individual in need thereof, comprising administering a therapeutically effective amount of a gallium-containing composition and an antibiotic substance to the individual, wherein the gallium-containing composition and the antibiotic substance act synergistically to treat the biofilm.

In another aspect, the invention provides a method for treating an orally-associated biofilm in an individual, comprising contacting the biofilm with a therapeutically effective amount of a gallium-containing composition. In one embodiment, the method comprises prevention of formation of a biofilm and/or prevention of spread of a biofilm to another site in the individual by administration of a prophylactically effective amount of the gallium-containing composition.

In one embodiment, the orally-associated biofilm is located on a tooth, for example, dental plaque located on a tooth. In other embodiments, the orally-associated biofilm is located on the tongue, oral mucosa, or gum. The gallium-containing composition may be formulated as a dentrifice, such as, for example, a toothpaste, a mouthwash composition, or a chewing gum, or as a paint, foam, gel, or varnish, for example, in a fluoride-containing composition for fluoride treatment.

In one embodiment, the invention provides a method for treating bacterial keratitis in an individual, comprising contacting a biofilm associated with bacterial keratitis in the eye of the individual with a therapeutically effective amount of a gallium-containing composition. The gallium-containing composition may be formulated as gallium-containing ophthalmic eye drops or contact lens solution.

In a further aspect, the invention provides gallium-containing compositions for treatment of a biofilm. In one embodiment, gallium-containing composition is formulated as a dentrifice, such as, for example, a toothpaste. In another embodiment, the gallium-containing composition is formulated as a mouthwash. In another embodiment, the gallium-containing composition is formulated as a gum for chewing. In another embodiment, the gallium-containing composition is formulated as ophthalmic eye drops. In another embodiment, the gallium-containing composition is formulated as contact lens solution. In another embodiment, the gallium-containing composition comprises at least one antibiotic substance, such as, for example, ciproflaxin, ampicillin, azithromycin, cephalosporin, doxycycline, fusidic acid, gentamycin, linezolid, levofloxacin, norloxacin, ofloxacin, rifampin, tetracycline, tobramycin, vancomycin, amikacin, deftazidime, cefepime, trimethoprim/sulfamethoxazole, piperacillin/tazobactam, aztreanam, meropenem, colistin, or chloramphenicol and optionally, a pharmaceutically acceptable carrier. In another embodiment, the gallium-containing composition comprises at least one antibiotic substance from a class of antibiotics including but not limited to aminoglycosides, carbacephem, carbapenems, first generation cephalosporins, second generatin cephalosporins, third generation cephalosporins, fourth generation cephalosporins, glycopeptides, macrolides, monobactam, penicillins, polypeptides, quinolones, sulfonamides, tetracyclines, lincosamides, and oxazolidinones.

In a still further aspect, the invention provides kits for treatment (including prevention) of a biofilm-associated infection. Kits of the invention comprise a gallium-containing composition and packaging. Kits may include instructions for use in treatment of a biofilm-associated infection. In some embodiments, kits include at least one antibiotic substance. In some embodiments, kits include a gallium-containing composition formulated as a dentrifice, such as a toothpaste, a mouthwash composition, or chewing gum composition, or as or as a paint, foam, gel, or varnish, for example, in a fluoride-containing composition for fluoride treatment. In some embodiments, kits include a gallium-containing composition formulated as ophthalmic eye drops or contact lens solution. In some embodiments, kits include a pharmaceutical composition comprising a gallium-containing composition and a pharmaceutically acceptable carrier.

### BRIEF DESCRIPTION OF THE DRAWINGS

FIG. 1 depicts biophotonic monitoring of the effect of gallium maltolate (300 mg/kg) and ciprofloxacin in a P. aeruginosa Xen 5 UTI mouse model. The viable counts in the catheter were determined immediately after removal of the catheter from the mouse bladder, and are shown on the right hand side of the figure (with open symbols corresponding to the treatment described for closed symbols at the top of the figure).

FIG. 2 shows real-time monitoring of the effect of gallium maltolate (300 mg/kg) and ciprofloxacin in a P. aeruginosa Xen 5 UTI mouse model. A representative animal from each group is shown.

FIG. 3 shows pharmacokinetic data for gallium maltolate dosing in female CF-1 mice.

FIG. 4 shows the results of scanning electron microscopy analysis of longitudinal sections of explanted catheters bearing Pseudomonas aeruginosa biofilms.

### DETAILED DESCRIPTION

The invention provides methods, compositions, and kits for treatment of biofilm-associated infections. In particular, gallium-containing compositions are administered in methods of the invention for treatment (including prophylaxis, therapy, and cure) of biofilm-associated infections in an individual in need thereof, optionally in conjunction with administration of one or more antibiotic substances or one or more nonantibiotic antimicrobial substances.

General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture ( J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies : a practical approach (P. Shepherd and C. Dean, eds., Oxford University Press, 2000); Using antibodies: a laboratory manual (E. Harlow and D. Lane (Cold Spring Harbor Laboratory Press, 1999); and The Antibodies (M. Zanetti and J. D. Capra, eds., Harwood Academic Publishers, 1995).

Definitions

Unless otherwise indicated, the invention is not limited to specific synthetic methods, analogs, substituents, pharmaceutical formulations, formulation components, modes of administration, or the like, as such may vary. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Thus, for example, reference to “a substituent” includes a single substituent as well as two or more substituents that may be the same or different, reference to “a compound” encompasses a combination or mixture of different compounds as well as a single compound, reference to “a pharmaceutically acceptable carrier” includes two or more such carriers as well as a single carrier, and the like.

The term “alkyl” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, preferably 1 to about 12 carbon atoms. The term “lower alkyl” intends an alkyl group of 1 to 6 carbon atoms. Preferred lower alkyl substituents contain 1 to 3 carbon atoms, and particularly preferred such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Preferred aryl groups contain 5 to 24 carbon atoms, and particularly preferred aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroato, as will be described in further detail infra. If not otherwise indicated, the term “aryl” includes unsubstituted, substituted, and/or heteroatom-containing aromatic substituents.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage, or substituent in which one or more carbon atoms are replaced with an atom other than carbon, e.g., nitrogen, oxygen, sulfur, phosphorus, germanium, or silicon, typically nitrogen, oxygen or sulfur, preferably nitrogen or oxygen. Similarly, the term “heteroalkyl” refers to an alkyl substituent that is heteroatom-containing, the term “heterocyclic” refers to a cyclic substituent that is heteroatom-containing, the terms “heteroaryl” and “heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

“Hydrocarbyl” refers to univalent hydrocarbyl radicals containing 1 to about 30 carbon atoms, preferably 1 to about 24 carbon atoms, more preferably 1 to about 18 carbon atoms, most preferably about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

By “substituted” as in “substituted alkyl,” “substituted aryl,” and the like, as alluded to in some of the aforementioned definitions, is meant that in the alkyl, aryl, or other moiety, at least one hydrogen atom bound to a carbon (or other) atom is replaced with one or more non-hydrogen substituents. Examples of such substituents include, without limitation: functional groups such as halo, hydroxyl, sulfhydryl, C₁-C₂₄ alkoxy, C₂-C₂₄ alkenyloxy, C₂-C₂₄ alkynyloxy, C₅-C₂₄ aryloxy, acyl (including C₂-C₂₄ alkylcarbonyl (—CO-alkyl) and C₆-C₂₄ arylcarbonyl (—CO-aryl)), acyloxy (—O-acyl), C₂-C₂₄ alkoxycarbonyl (—(CO)—O-alkyl), C₆-C₂₄ aryloxycarbonyl (—(CO)—O-aryl), halocarbonyl (—CO)—X where X is halo), C₂-C₂₄ alkylcarbonato (—O—(CO)—O-alkyl), C₆-C₂₄ arylcarbonato (—O—(CO)—O-aryl), carboxy (—COOH), carboxylato (—COO—), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-(C₆-C₂₄ aryl)-substituted carbamoyl (—(CO)—NH-aryl), di-(C₆-C₂₄ aryl)-substituted carbamoyl (—(CO)—N(aryl)₂), di-N—(C₁-C₂₄ alkyl), N—(C₆-C₂₄ aryl)-substituted carbamoyl, thiocarbamoyl (—(CS)—NH₂), carbamido (—NH—(CO)—NH₂), cyano(—C≡N), isocyano (—N⁺≡C⁻), cyanato (—O—C≡N), isocyanato (—O—N⁺≡C⁻), isothiocyanato (—S—C≡N), azido (—N═N⁺═N⁻), formyl (—(CO)—H), thioformyl (—(CS)—H), amino (—NH₂), mono-(C₁-C₂₄ alkyl)-substituted amino, di-(C₁-C₂₄ alkyl)-substituted amino, mono-(C₅-C₂₄ aryl)-substituted amino, di-(C₅-C₂₄ aryl)-substituted amino, C₂-C₂₄ alkylamido (—NH—(CO)-alkyl), C₆-C₂₄ arylamido (—NH—(CO)-aryl), imino (—CR═NH where R=hydrogen, C₁-C₂₄ alkyl, C₅-C₂₄ aryl, C₆-C₂₄ alkaryl, C₆-C₂₄ aralkyl, etc.), alkylimino (—CR═N(alkyl), where R=hydrogen, C₁-C₂₄ alkyl, C₅-C₂₄ aryl, C₆-C₂₄ alkaryl, C₆-C₂₄ aralkyl, etc.), arylimino (—CR═N(aryl), where R=hydrogen, C₁-C₂₄ alkyl, C₅-C₂₄ aryl, C₆-C₂₄ alkaryl, C₆-C₂₄ aralkyl, etc.), nitro (—NO₂), nitroso (—NO), sulfo (—SO₂—OH), sulfonato (—SO₂—O⁻), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₄ arylsulfinyl (—(SO)-aryl), C₁-C₂₄ alkylsulfonyl (—SO₂-alkyl), C₅-C₂₄ arylsulfonyl (—SO₂-aryl), phosphono (—P(O)(OH)₂), phosphonato (—P(O)(O⁻)₂), phosphinato (—P(O)(O⁻)), phospho (—PO₂), and phosphino (—PH₂); and the hydrocarbyl moieties C₁-C₂₄ alkyl (preferably C₁-C₁₈ alkyl, more preferably C₁-C₁₂ alkyl, most preferably C₁-C₆ alkyl), C₂-C₂₄ alkenyl (preferably C₂-C₁₈ alkenyl, more preferably C₂-C₁₂ alkenyl, most preferably C₂-C₆ alkenyl), C₂-C₂₄ alkynyl (preferably C₂-C₁₈ alkynyl, more preferably C₂-C₁₂ alkynyl, most preferably C₂-C₆ alkynyl), C₅-C₂₄ aryl (preferably C₅-C₁₄ aryl), C₆-C₂₄ alkaryl (preferably C₆-C₁₈ alkaryl), and C₆-C₂₄ aralkyl (preferabl C₆-C₁₈ aralkyl).

In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated.

When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

“Optional” or “optionally” means that the subsequently described circumstance may or may not occur, so that the description includes instances where the circumstance occurs and instances where it does not. For example, the phrase “optionally substituted” means that a non-hydrogen substituent may or may not be present on a given atom, and, thus, the description includes structures wherein a non-hydrogen substituent is present and structures wherein a non-hydrogen substituent is not present. Similarly, the phrase an “optionally present” bond as indicated by a dotted or dashed line—means that a bond may or may not be present.

When referring to a compound of the invention as an active agent, applicants intend the term “compound” or “active agent” to encompass not only the specified molecular entity but also its pharmaceutically acceptable, pharmacologically active analogs, including, but not limited to, salts, esters, amides, hydrates, solvates, prodrugs, conjugates, active metabolites, and other such derivatives, analogs, and related compounds.

The terms “treating” and “treatment” as used herein refer to causing a reduction in severity and/or frequency of symptoms, elimination of symptoms and/or underlying cause, prevention of the occurrence of symptoms and/or their underlying cause, and/or improvement or remediation of damage. Thus, “treating” a patient with a compound of the invention includes prevention of a particular disorder or adverse physiological event in a susceptible individual, as well as management of a clinically symptomatic individual to inhibit or cause regression of a disorder or disease. Treatment can include prophylaxis, therapy, or cure. For example, treatment of a biofilm encompasses prevention of formation of a biofilm in a patient susceptible to development of a biofilm (e.g., at a higher risk, as a result of genetic predisposition, environmental factors, predisposing diseases or disorders, or the like), as well as treatment of a patient with a biofilm by inhibiting, or causing regression of, the disease.

By the terms “effective amount” refers to the amount of a gallium-containing composition that provides gallium in a sufficient amount to render a desired treatment outcome. An effective amount may be comprised within one or more doses, i.e., a single dose or multiple doses may be required to achieve the desired treatment endpoint. A “therapeutically effective amount” refers to an amount of gallium-containing composition sufficient to produce a desired therapeutic outcome (e.g., reduction of severity of, or elimination of, a biofilm). A “prophylactically effective amount” refers to an amount of gallium-containing composition sufficient to prevent or reduce severity of a future biofilm when administered to an individual who is susceptible and/or who may develop a biofilm.

The term “controlled release” refers to a drug-containing formulation or fraction thereof in which release of the drug is not immediate, i.e., with a “controlled release” formulation, administration does not result in immediate release of the drug into an absorption pool. The term is used interchangeably with “nonimmediate release” as defined in Remington: The Science and Practice of Pharmacy, Nineteenth Ed. (Easton, PA: Mack Publishing Company, 1995). In general, the term “controlled release” as used herein includes sustained release and delayed release formulations.

By “pharmaceutically acceptable” is meant a material that is not biologically or otherwise undesirable, i.e., the material may be incorporated into a pharmaceutical composition administered to a patient without causing any significant undesirable biological effects or interacting in a deleterious manner with any of the other components of the composition in which it is contained. When the term “pharmaceutically acceptable” is used to refer to a pharmaceutical carrier or excipient, it is implied that the carrier or excipient has met the required standards of toxicological and manufacturing testing or that it is included on the Inactive Ingredient Guide prepared by the U.S. Food and Drug administration.

An “individual” refers to a vertebrate, typically a mammal, commonly a human.

Methods of Use

Methods are provided for administration of a gallium-containing composition to an individual in need of treatment for a biofilm-associated infection. Methods of the invention include prophylaxis, therapy, or cure of a biofilm-associated infection. Methods include administration of one or more unit doses of a gallium-containing composition in a therapeutically or prophylactically effective amount. In methods of the invention, gallium-containing compositions are generally administered in a pharmaceutically acceptable carrier. In some embodiments, the methods comprise treatment of a nosocomial biofilm-associated infection.

In methods of the invention, a gallium-containing composition is administered to an individual in a therapeutically or prophylactically effective amount for treatment of an existing biofilm-associated infection or prevention of establishment of a biofilm-associated infection in the individual. In some embodiments, spread of a biofilm-associated infection to another site in the individual is inhibited. In various embodiments, the gallium-containing composition may be administered parenterally, orally, locally, or topically.

A gallium-containing composition may be administered in a single daily dose or in multiple doses, e.g., 2, 3, 4, or more doses, per day. Generally, when administered to a human, the gallium-containing composition is administered to provide a total daily amount of gallium of about 2 mg to about 800 mg. In some embodiments, the total daily amount of gallium administered is about 2 mg to about 15 mg, about 8 mg to about 40 mg, about 15 mg to about 80 mg, about 40 mg to about 160 mg, about 150 mg to about 325 mg, or about 300 mg to about 500 mg, about 500 mg to about 700 mg, or about 600 mg to about 800 mg.

The actual dosage may vary depending upon the gallium compound administered and the dosage can be selected so as to provide a predetermined amount of Ga(III) to be delivered per kilogram of patient weight. For example, some methods of the invention may involve administering a gallium compound that provides about 0.1 to about 20 mg Ga(III)/kg, often about 1 to about 12 mg Ga(III)/kg.

In some methods of the invention involving systemic administration (e.g., parenteral, oral), a gallium-containing composition is administered to an individual in an amount sufficient to provide a therapeutically or prophylactically effective serum gallium level for prevention or treatment of a biofilm. In one embodiment, the gallium-containing composition is administered in a unit dose that results in a gallium serum level, at about 24 hours following administration, of at least about 10 ng/mL. In various embodiments, a therapeutically or prophylactically effective serum level of gallium, at about 24 hours following administration, is at least any of about 10, 25, 50, 100, 200, 500, 1000, 2000, 3000, 4000, 5000, 6000, or 7000 ng/mL A therapeutically or prophylactically effective serum level is typically reached within about 1, 2, 6, 12, 24, 48, or 72 hours following administration of the gallium-containing composition to the individual.

In some embodiments, methods of the invention comprise administration of a therapeutically effective gallium-containing composition to an individual in need thereof for treatment of a biofilm-associated infection in the bladder, kidney, heart, middle ear, sinuses, skin, lung, a joint, subcutaneous tissue, soft tissue, vascular tissue, and/or the eye. In some embodiments, the biofilm-associated infection is at least at one site other than the lung and/or the skin. In one embodiment, a therapeutically effective amount of a gallium-containing composition is administered to an individual in need thereof for treatment of a biofilm-associated urinary tract infection. In another embodiment, a therapeutically effective amount of a gallium-containing composition is administered to an individual in need thereof for treatment of biofilm-associated chronic bacterial vaginosis. In another embodiment, a therapeutically effective amount of gallium is administered to an individual in need thereof for treatment of a biofilm-associated prostatitis. In another embodiment, a therapeutically effective amount of gallium is administered to an individual in need thereof for treatment of a biofilm-associated bacterial infection stemming from diabetes, such as a diabetic skin ulcer. In another embodiment, a therapeutically effective amount of gallium is administered to an individual in need thereof for treatment of a pressure ulcer. In another embodiment, a therapeutically effective amount of gallium is administered to an individual in need thereof for treatment of a biofilm-associated venous catheter-associated ulcer. In another embodiment, a therapeutically effective amount of gallium is administered to an individual in need thereof for treatment of a biofilm-associated surgical wound (e.g., a surgical site infection).

In one embodiment, the biofilm is in the lung of an individual wherein the individual does not have cystic fibrosis. In one embodiment, the biofilm is in the lung of an individual wherein the biofilm does not comprise Pseudomonas aeruginosa. Examples of lung infections treatable by the methods of the invention include, but are not limited to, pulmonary actinomycosis, Nocardia infection, a lung abscess, infectious bacterial bronchitis, and bacterial pneumonia (for example, comprising Streptococcus pneumoniae, H. influenzae, Klebsiella, Staphylococcus aureus, Legionella pneumophila, Escherichia coli, Pseudomonas, Enterobacter, or Serratia.

In one embodiment, the biofilm is on the skin of an individual wherein the skin does not comprise a burn wound. Examples of skin infections treatable by methods of the invention include, but are not limited to, bacterial skin infections, Kawasaki disease, Pseudofolliculitis barbae, Sarcoidosis, Scalp folliculitis, diabetic ulcers, and pressure ulcers. In one embodiment, the biofilm is below the surface of the skin, in subcutaneous tissue, such as a deep tissue wound or a surgical site infection.

In some embodiments, methods of the invention further comprise administration of one or more unit doses of an antibiotic substance, including, but not limited to, ciproflaxin, ampicillin, azithromycin, cephalosporin, doxycycline, fusidic acid, gentamycin, linezolid, levofloxacin, norfloxacin, ofloxacin, rifampin, tetracycline, tobramycin, vancomycin, amikacin, deftazidime, cefepime, trimethoprim/sulfamethoxazole, piperacillin/tazobactam, aztreanam, meropenem, colistin, or chloramphenicol. In some embodiments, methods of the invention further comprise administration of one or more unit doses of an antibiotic substance from an antibiotic class including, but not limited to, aminoglycosides, carbacephem, carbapenems, first generation cephalosporins, second generatin cephalosporins, third generation cephalosporins, fourth generation cephalosporins, glycopeptides, macrolides, monobactam, penicillins, polypeptides, quinolones, sulfonamides, tetracyclines, lincosamides, and oxazolidinones.

The gallium-containing composition and antibiotic substance may act synergistically or additively to treat the biofilm-associated infection. The gallium-containing composition and the antibiotic substance may be administered simultaneously in the same or separate compositions, or sequentially.

In some embodiments, methods of the invention further comprise administration of one or more unit doses of a nonantibiotic antimicrobial substance, including, but not limited to, sertraline, racemic and stereoisomeric forms of thioridazine, benzoyl peroxide, taurolidine, and hexitidine. The gallium-containing composition and nonantibiotic antimicrobial substance may act synergistically or additively to treat the biofilm-associated infection. The gallium-containing composition and the nonantibiotic antimicrobial substance may be administered simultaneously in the same or separate compositions, or sequentially.

In some embodiments, methods of the invention comprise treatment of an orally-associated biofilm in an individual, comprising contacting an oral surface with a therapeutically effective amount of a gallium-containing composition. Some methods of the invention comprise prevention of an orally-associated biofilm by administration of a prophylactically effective amount of a gallium-containing composition to an individual. The orally-associated biofilm may be, for example, dental plaque, and the gallium-containing composition may be formulated as a dentrifice, such as toothpaste, for treatment or prevention of dental plaque. In other embodiments, the biofilm may be located on the tongue, oral mucosa, or gums. In some embodiments, the gallium-containing composition is formulated as a mouthwash. In some embodiments, the gallium-containing composition is formulated as a paint, foam, gel, or varnish, for example, in a fluoride-containing composition. In one embodiment, the gallium-containing composition is in the form or a gel or foam in a mouthguard that a patient wears for several minutes for fluoride treatment.

In the methods described herein, two or more gallium-containing compositions may be co-administered. In some embodiments, one or more gallium-containing compositions may be co-administered with one or more therapeutically beneficial substances, such as, for example, one or more antibiotic substances, iron-chelating agents, e.g., desferrioxamine (Olivieri et al. (1997) Blood 89(3):739-61), or quorum-sensing drugs, e.g., RNAIII-ihibiting peptide (RIP) (Dell'Acqua et al. (2004) J Infect Dis 190:318-20).

Gallium-Containing Compositions

In accordance with methods of the invention as described herein, a gallium-containing composition can be administered that comprises, for example, a coordination complex of gallium (III), a salt of gallium (III), an inorganic compound of gallium (III) other than a salt, or protein-bound gallium (III). For administration to an individual, a pharmaceutical composition may be administered comprising a gallium-containing composition as described herein and a pharmaceutically acceptable carrier.

Gallium (III) coordination complexes are complexes that comprise a Ga(III) center coordinated to one or more ligands. Coordination complexes of gallium (III) include, without limitation, gallium (III) complexes of an N-heterocycle (such as tris (8-quinolinolato) gallium (III)), gallium (III) complexes with hydroxypyrones, including neutral 3:1 gallium complexes of a 3-hydroxy-4-pyrone (such as gallium maltolate), gallium complexes with hydroxypyridinones or substituted hydroxypyridinones, gallium porphyrins (such as gallium (III) protoporphyrin IX), pyridoxal isonicotinoyl hydrazone gallium (III), and gallium salt complexes of polyether acids. Such coordination complexes include, but are not limited to, those comprising three bidentate ligands or one tridentate ligand. Bidentate ligands are each coordinated to the gallium (III) center through two oxygen, nitrogen, or sulfur atoms; the two coordinating atoms may be the same or different. Similarly, tridentate ligands are coordinated to the gallium (III) center through three oxygen, nitrogen, or sulfur atoms; the three coordinating atoms may be the same or different. The coordinating ligands may all be the same or there may be a mixture of different ligands.

Bidentate ligands may be, for example, unsubstituted hydroxypyrone, or hydroxypyrone substituted at the 2-, 5-, and/or 6-positions with a C₁-C₆ alkyl group. In particular, bidentate ligands can be 2-substituted or 5-substituted hydroxypyrones, such as 3-hydroxy-2-methyl-4-pyrone (maltol) and 3-hydroxy-2-ethyl-4-pyrone (ethyl maltol). Other examples of bidentate ligands are unsubstituted hydroxypyridinones, or hydroxypyridinones substituted at the 2-, 5-, and/or 6-positions with a C₁-C₆ alkyl group. An example of a tridentate ligand is pyridoxal isonicotinoyl hydrazone.

Further, the ligands may be of the formula Ar—O—, wherein Ar is an aryl, heteroaryl, substituted aryl, or substituted heteroaryl group. For example, the Ar group may be an optionally substituted heteroaryl group such as the anion of 8-hydroxyquinoline.

The ligands also may be selected from carboxylate ligands having the structure R—(CO)—O—, where R is hydrocarbyl, a substituted hydrocarbyl, a heteroatom-containing hydrocarbyl, or a substituted heteroatom-containing hydrocarbyl.

In one embodiment, a gallium composition suitable for use in accordance with the methods of the invention comprises a gallium complex of a 3-hydroxy-4-pyrone, such as, for example, gallium maltolate. The synthesis of such complexes and preparations of the complexes in pharmaceutical formulations, have been described, for example, in U.S. Pat. Nos. 5,258,376, 5,574,027, 5,883,088, 5,968,922, 5,981,518, 5,998,397, 6,004,951, 6,048,851, and 6,087,354.

Gallium salts include both inorganic and organic salts. Examples of inorganic salts and related inorganic compounds include, but are not limited to, gallium chloride, gallium nitrate, gallium sulfate, gallium carbonate, and gallium phosphate. Hydrated and solvated forms of these salts are included. Examples of organic salts include, but are not limited to, gallium acetate, gallium citrate, gallium formate, gallium hydroxamate, gallium oxalate, gallium glutamate, gallium palmitate, and gallium tartrate, as well as their hydrated and solvated forms. Examples of inorganic gallium compounds other than gallium salts are gallium oxide and gallium oxide hydroxide, as well as their hydrated and solvated forms.

Other compositions suitable for use in the methods of the invention include peptides and proteins containing bound gallium. Examples of such compositions include gallium-lactoferrin and gallium-transferrin. In some embodiments, the protein is derived from the species to be treated. In some embodiments, protein-bound gallium-containing compositions are conjugated with one or more other active agents. An example of such a conjugate is gallium-transferrin-doxorubicin conjugate.

Bioflims

A “biofilm” as used herein refers to an aggregate of microorganisms with an extracellular matrix that facilitates adhesion to, and colonization and growth of the aggregate on a surface, such as an internal or external tissue or organ. Biofilms can be comprised of bacteria, fungi, yeast, protozoa, or other microorganisms. Bacterial biofilms are typically characterized by a high resistance to antibiotics, often up to 1,000-times greater resistance than the same bacteria not growing in a biofilm.

In some embodiments, methods of the invention comprise treatment (including prevention) of a biofilm on an internal organ or tissue, such as the bladder, kidney, heart, middle ear, sinuses, the lung, a joint, the eye, or an external tissue, such as the skin. In some embodiments, methods of the invention comprise treatment (including prevention) of a biofilm on an oral surface such as teeth, tongue, oral mucosa, or gums. Methods of the invention may be used to treat a biofilm-associated condition such as a soft-tissue infection, chronic sinusitis, endocarditis, osteomyelitis, urinary tract infection, chronic bacterial vaginosis, dental plaque or halitosis, bacterial keratitis, or prostatitis.

Bacterial biofilms may be formed by both Gram-positive and Gram-negative bacterial species. Examples of Gram-positive bacteria that are capable of forming biofilms include, but are not limited to, Staphylococcus aureus, coagulase negative staphylococci such as Staphylococcus epidermis, Streptococcus pyogenes (Group A), Streptococcus species (viridans group), Streptococcus agalactiae (group B), S. bovis, Streptococcus (anaerobic species), Streptococcus pneumoniae, and Enterococcus species. Other Gram-positive bacilli include Bacillus anthracis, Corynebacterium diptheriae, and Corynebacterium species which are diptheroids (aerobic and anaerobic), Listeria monocytogenes, Clostridium tetani, and Clostridium difficile. Examples of Gram-negative bacteria that are capable of forming biofilms include, but are not limited to, Escherichia coli, Enterobacter species, Proteus mirablis and other species, Pseudomonas aeruginosa, Klebsiella pneumoniae, Salmonella, Shigella, Serratia, and Campylobacterjejuni, Neisseria, Branhamella catarrhalis, and Pasteurella.

Other organisms capable of forming biofilms include, but are not limited to dermatophytes (e.g, Microsporum species such as Microsporum canis, Trichophyton species such as Trichophyton rubrum and Trichophyton mentagrophytes), yeasts (e.g., Candida albicans, Candidaparapsilosis, Candida glabrata, Candida tropicalis, and other Candida species including drug resistant Candida species), Epidermophytonfloccosum, Malasseziafuurfur (Pityropsporon orbiculare, Pityropsporon ovale) Cryptococcus neoformans, Aspergillusfumigatus and other Aspergillus species, Zygomycetes (Rizopus, Mucor), hyalohyphomycosis (Fusarium species), Paracoccidiodes brasiliensis, Blastmyces dermatitides, Histoplasma capsulatum, Coccidiodes immitis, Sporothrix schenckii, and Blastomyces.

Modes of Administration

Administration of gallium-containing compounds in accordance with the methods of the invention may be via any route that provides a desired therapeutic or prophylactic effect, e.g., reduction, elimination, or prevention of a biofilm.

In some embodiments, one or more gallium-containing composition is formulated for local or topical administration for treatment or prevention of an orally-associated biofilm. Administration may be, for example, to an oral site such as the teeth, tongue, oral mucosa, or gums. In some embodiments, the gallium-containing composition is formulated as a dentrifice, for example, a toothpaste composition. In some embodiments, the gallium-containing composition is formulated as a mouthwash. In some embodiments, the gallium-containing composition is formulated as a paint, foam, gel, or varnish, for example, in a fluoride-containing composition.

A toothpaste composition may optionally contain, in addition to one or more gallium-containing compositions, one or more abrasives (e.g., alumina, hydrated silica, dicalcium phosphate, salt, pumice, kaolin, bentonite, calcium carbonate, sodium bicarbonate, calcium pyrophosphagae), one or more decay prevention components (e.g., sodium monofluorophosphate, stannous fluoride, sodium fluoride, xylitol), one or more antibacterial agents (e.g., triclosan, sanguinaria extract, baking soda, zinc citrate trihydrate, polyphenols, stannous fluoride), one or more tartar control agents (e.g., tetrasodium pyrophosphate, Gantrez S-70, sodium tri-polyphosphate), one or more enzymes to enhance antibacterial properties of saliva (e.g., glucose oxidase, lactoperoxidase, lysozyme), one or more desensitizing agents (e.g., potassium nitrate, strontium chloride, sodium citrate), one or more coloring agents, one or more detergents (e.g., sodium lauryl sulfate, sodium lauroyl sarcosinate, sodium N-lauryl sarcosinate, dioctyl sodium sulfosuccinate, sodium stearyl funarate, sodium stearyl lactate, sodium lauryl sulfoacetate), one or more flavorings (e.g., mint, menthol, peppermint, spearmint, cinnamon, wintergreen, fennel), one or more humectants (e.g., sorbitol, pentatol, glycerol glycerin, propylene glycol, polyethylene glycol, water, xylitol, PEG 8 (polyoxyethylene glycol esters), PPG (polyoxyethylene ethers), one or more thickeners (e.g., carrageenan, cellulose gum, xanthan gum, gum Arabic, sodium carboxymethyl cellulose, cellulose ethers, sodium alginate, carbopols, silica thickeners, sodium aluminum silicates, clays), one or more preservatives (e.g., sodium benzoate, methyl paraben, ethyl paraben), one or more sweeteners (e.g., calcium or sodium saccharin, aspartame), water, one or more whiteners (e.g., peroxide, citroxain, titanium dioxide), and/or one or more beneficial agents (e.g., stabilized chorine dioxide, mellaleuca, neem, CPP-ACP).

A mouthwash composition may optionally contain, in addition to one or more gallium-containing compositions, one or more anti-bacterial compounds (e.g., quaternary ammonium compounds, boric acid, benzoic acid), one or more phenolic compounds, one or more flavoring agents (e.g., saccharin or glycerin), one or more astringents (e.g., zinc chloride), ethyl alcohol (typically 18-26% in water), one or more buffers, one or more decay prevention components (e.g., sodium fluoride, stannous fluoride), and/or one or more anti-plaque components (e.g., chlorhexidine, heavy metal salts, sanguinaria).

In some embodiments, one or more gallium-containing composition is formulated for treatment or prevention of bacterial keratitis. The gallium-containing composition may be formulated as ophthalmic eye drops or a contact lens cleaning or wetting solution. In one embodiment, the composition may be administered topically to the eye in ophthalmic eye drops.

In some embodiments, one or more gallium-containing composition is administered in a pharmaceutical composition that comprises a unit dose of the gallium-containing composition(s) and a pharmaceutically acceptable carrier. For example, administration may be oral or parenteral (e.g., intravenous, subcutaneous, intramuscular, transdermal, dermal, transmucosal (including buccal, nasal, rectal, sublingual, and vaginal), by inhalation, or via an implanted reservoir in a dosage form).

In some embodiments, a gallium containing composition, such as for example, a coordination complex of gallium (III), e.g., gallium maltolate, is administered orally. In some embodiments, the coordination complex is a complex of gallium (III) and 3-hydroxy-2-methyl-4-pyrone. In some embodiments, this complex is administered orally once per day to achieve and maintain a therapeutically or prophylactically effective serum level of gallium, for example, a serum level of at least about 10, 25, 50, 100, 200, 500, 1000, 2000, 3000, 4000, 5000, 6000, or 7000 ng/ml.

Depending on the intended mode of administration, the pharmaceutical formulation may be a solid, semi-solid, or liquid, such as, for example, a tablet, a capsule, a caplet, a liquid, a suspension, an emulsion, a gel, an ointment, a suppository, granules, pellets, beads, a powder, or the like, preferably in unit dosage form suitable for single administration of a precise dosage. Suitable pharmaceutical compositions and dosage forms may be prepared using conventional methods known to those in the field of pharmaceutical formulation and described in the pertinent texts and literature, e.g., in Remington: The Science and Practice of Pharmacy (Easton, Pa.: Mack Publishing Co., 1995). For those compounds that are orally active, oral dosage forms are generally preferred, and include tablets, capsules, caplets, solutions, suspensions, and syrups, and may also comprise a plurality of granules, beads, powders, or pellets that may or may not be encapsulated. Preferred oral dosage forms are tablets and capsules.

Tablets may be manufactured using standard tablet processing procedures and equipment. Direct compression and granulation techniques are preferred. In addition to the active agent, tablets will generally contain inactive, pharmaceutically acceptable carrier materials such as binders, lubricants, disintegrants, fillers, stabilizers, surfactants, coloring agents, and the like. Binders are used to impart cohesive qualities to a tablet, and thus ensure that the tablet remains intact. Suitable binder materials include, but are not limited to, starch (including corn starch and pregelatinized starch), gelatin, sugars (including sucrose, glucose, dextrose, and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, microcrystalline cellulose, ethyl cellulose, hydroxyethyl cellulose, and the like), and Veegum. Lubricants are used to facilitate tablet manufacture, promoting powder flow and preventing particle capping (i.e., particle breakage) when pressure is relieved. Useful lubricants are magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers, as well known in the art, are used to inhibit or retard drug decomposition reactions that include, by way of example, oxidative reactions.

Capsules are also a preferred oral dosage form, in which case the active agent-containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals.

Oral dosage forms, whether tablets, capsules, caplets, or particulates, may, if desired, be formulated so as to provide for gradual, sustained release of the active agent over an extended time period. Generally, as will be appreciated by those of ordinary skill in the art, sustained release dosage forms are formulated by dispersing the active agent within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

Preparations according to this invention for parenteral administration include sterile aqueous and nonaqueous solutions, suspensions, and emulsions. Injectable aqueous solutions contain the active agent in water-soluble form. Examples of nonaqueous solvents or vehicles include fatty oils, such as olive oil and corn oil, synthetic fatty acid esters, such as ethyl oleate or triglycerides, low molecular weight alcohols such as propylene glycol, synthetic hydrophilic polymers such as polyethylene glycol, liposomes, and the like. Parenteral formulations may also contain adjuvants such as solubilizers, preservatives, wetting agents, emulsifiers, dispersants, and stabilizers, and aqueous suspensions may contain substances that increase the viscosity of the suspension, such as sodium carboxymethyl cellulose, sorbitol, and dextran. Injectable formulations are rendered sterile by incorporation of a sterilizing agent, filtration through a bacteria-retaining filter, irradiation, or heat. They can also be manufactured using a sterile injectable medium. The active agent may also be in dried, e.g., lyophilized, form that may be rehydrated with a suitable vehicle immediately prior to administration via injection.

The compounds of the invention may also be administered through the skin using conventional transdermal drug delivery systems, wherein the active agent is contained within a laminated structure that serves as a drug delivery device to be affixed to the skin. In such a structure, the drug composition is contained in a layer, or “reservoir,” underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during drug delivery. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. Transdermal drug delivery systems may in addition contain a skin permeation enhancer.

In addition to the formulations described previously, the compounds may also be formulated as a depot preparation for controlled release of the active agent, preferably sustained release over an extended time period. These sustained release dosage forms are generally administered by implantation (e.g., subcutaneously or intramuscularly or by intramuscular injection).

Administration may be rectal or vaginal, preferably using a suppository that contains, in addition to the active agent, excipients such as a suppository wax.

Formulations for nasal or sublingual administration are also prepared with standard excipients well known in the art. The pharmaceutical compositions of the invention may also be formulated for inhalation, e.g., as a solution in saline, as a dry powder, or as an aerosol.

Administration of Gallium in Combination with at Least One Antibiotic Substance

In some embodiments, one or more gallium-containing compositions as described above are administered in conjunction with one or more antibiotic substances. The gallium-containing composition(s) may be administered simultaneously or sequentially with the antibiotic substance(s). For simultaneous administration, the gallium-containing composition and the antibiotic substance may be administered in the same or separate pharmaceutical compositions. One or multiple unit doses of a gallium-containing composition and one or multiple unit doses of an antibiotic substance may be administered in a method for treatment of a biofilm as described herein.

Antibiotic substances that may be used in methods of the invention include, but are not limited to, ciproflaxin, ampicillin, azithromycin, doxycycline, fusidic acid, gentamycin, linezolid, levoflaxacin, norfloxacin, ofloxacin, rifampin, tetracycline, or tobramycin, either alone or in combination. If two or more antibiotic substances are used in combination, they may be administered either simultaneously or sequentially.

In some embodiments, the gallium-containing composition(s) and antibiotic substance(s) may act synergistically to treat a biofilm-associated infection. In other embodiments, the gallium-containing composition(s) and antibiotic substance(s) may act additively to treat a biofilm-associated infection.

An antibiotic substance may be administered by any route that provides a desired therapeutic or prophylactic effect, e.g., reduction, elimination, or prevention of a biofilm, in conjunction with administration of a gallium-containing composition. In some embodiments, the antibiotic substance is administered parenterally, e.g., intramuscularly, intravenously, subcutaneously, intraperitoneally, or intrathecally. In some embodiments, the antibiotic substance is administered orally. In some embodiments, the antibiotic substance is administered topically or locally.

Bacterial Keratitis

Infections bacteria that create a breakdown of the comeal epithelium may cause bacterial keratitis, which is a sight-threatening process. Some virulent bacteria that may penetrate the intact epithelium (for example, Neisseria gonorrhoeae) also may result in bacterial keratitis.

Bacterial keratitis can progress rapidly and complete corneal destruction may occur by 24-48 hours with some of the more virulent bacteria. Corneal ulceration, stromal abscess formation, surrounding corneal edema, and anterior segment inflammation are characteristic of this disease.

The most common groups of bacteria responsible for bacterial keratitis are Streptococcus, Pseudomonas, Enterobacteriaceae (including Klebsiella, Enterobacter, Serratia, and Proteus), and Staphylococcus species. Up to 20% of cases of fungal keratitis (particularly candidiasis) are complicated by bacterial co-infection.

The most common cause of trauma to the corneal epithelium and the main risk factor for bacterial keratitis is the use of contact lenses, particularly extended-wear contact lenses.

The current standard of care for bacterial keratitis begins with broad-spectrum antibiotics if no organisms are identified is tobramycin (14 mg/ml), 1 drop every hour, alternating with fortified cefazolin (50 mg/ml), 1 drop every hour. If the corneal ulcer is small, peripheral, and no impending perforation is present, intensive monotherapy with fluoroquinolones is an alternative treatment.

Gallium maltolate may be used in the treatment of bacterial keratitis, by directly inhibiting bacterial growth, killing the bacteria, and/or by facilitating the destruction of the biofilm. Biofilm encapsulation prevents the complete eradication of the infection by standard antibiotic treatment, and can lead to flare-ups of the infection after antibiotic therapy is terminated. Gallium maltolate may break up the matrix of the corneal biofilm, thus rendering the infectious bacteria sensitive to antibiotic treatment.

Ophthalmic drops and contact lens cleaning and reconditioning solutions need to be maintained under strict sterility in order to avoid causing sight-threatening corneal infections. Several different preservatives have been used to restrain microorganism growth in ophthalmic solutions. These include 0.001% polyhexanide, 3% microfiltered hydrogen peroxide, sodium perborate stabilized with phosphoric acid, and sodium benzoate. Frequently, these solutions are preservative-free because many patients are preservative sensitive. Gallium maltolate may be used as a preservative in ophthalmic solutions to prevent bacterial survival and growth.

Compositions

The invention provides compositions for treatment (e.g., prophylaxis, therapy, or cure) of a biofilm and/or prevention of spread of a biofilm-associated infection to another site in the body.

In one embodiment, the invention provides a pharmaceutical composition comprising a gallium-containing composition, and a pharmaceutically acceptable carrier, wherein the gallium-containing composition is in a therapeutically effective amount to treat a biofilm or a prophylactically effective amount to prevent formation of a biofilm. In some embodiments, the pharmaceutical composition further comprises an antibiotic or nonantibiotic antimicrobial substance, in an amount effective to act synergistically or additively with the gallium-containing composition to treat an existing biofilm, prevent formation of a biofilm, or prevent spread of a biofilm-associated infection to another site in the body.

In another embodiment, the invention provides a composition for treatment or prevention of an orally-associated biofilm, for example, on the teeth, tongue, oral mucosa, or gums. In some embodiments, the composition comprises a gallium-containing composition formulated as a dentrifice, for example, a toothpaste. In other embodiments, the composition comprises a gallium-containing composition formulated as mouthwash. In other embodiments, the composition comprises a gallium-containing composition formulated as a paint, foam, gel, or varnish for dental use, for example, a composition for fluoride treatment.

In another embodiment, the invention provides a composition for treatment or prevention of bacterial keratitis. In some embodiments, the composition comprises a gallium-containing composition formulated as ophthalmic eye drops.

In another embodiment, the invention provides a contact lens solution that contains gallium as a preservative and/or anti-biofilm agent, for prevention of bacterial growth and/or biofilm formation in the solution.

Kits

The invention provides kits for use in a method of treatment (e.g., prophylaxis, therapy, or cure) of a biofilm and/or prevention of spread of a biofilm-associated infection to another site in the body as described herein. In some embodiments, kits contain a gallium-containing composition, generally formulated as a pharmaceutical composition. The kits may also optionally contain an antibiotic substance. In some embodiments, kits contain a gallium-containing composition for treatment of an orally-associated biofilm, such as a dentrifice (e.g., toothpaste), chewing gum, or mouthwash composition, or a gel, foam, paint, or varnish, for example, in a fluoride-containing composition for fluoride treatment. In some embodiments, kits contain a gallium-containing composition for treatment of bacterial keratitis, such as ophthalmic eye drops or contact lens solution. In some embodiments, kits contain a gallium-containing contact lens solution, wherein gallium is provided as a preservative to prevent bacterial growth and/or biofilm formation in the solution. Instructions may be included, providing information to a health care provider, patient, or consumer regarding use of the gallium-containing composition for treatment of a biofilm in accordance with the methods described herein. Instructions may be provided in printed form or in the form of an electronic medium such as a floppy disc, CD, or DVD, or in the form of a website address where such instructions may be obtained.

Suitable packaging is provided. As used herein, “packaging” refers to a solid matrix or material customarily used in a system and capable of holding within fixed limits a gallium-containing composition suitable for administration to an individual. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

In some embodiments, the kits contain a gallium-containing composition formulated in dosage forms each a unit dosage of the gallium-containing composition and a pharmaceutically acceptable carrier, wherein the unit dosage provides a therapeutically or prophylactically effective amount of gallium sufficient to treat a biofilm in an individual. The dosage forms may optionally be separately sealed and individually removable. The kits may optionally further include at least one antibiotic substance or nonantibiotic antimicrobial substance, optionally formulated in one or more dosage forms each containing a pharmaceutically acceptable carrier and a unit dosage of the antibiotic or nonantibiotic antimicrobial substance, wherein the unit dosage provides a therapeutically or prophylactically effective amount of the antibiotic substance sufficient to treat a biofilm in an individual in conjunction with administration of a gallium-containing composition as described herein, wherein the antibiotic or nonantibiotic antimicrobial substance optionally acts synergistically or additively with the gallium-containing composition to treat the biofilm. In some embodiments, the gallium-containing composition in the kit is in an orally active form, the pharmaceutically acceptable carrier is suitable for oral drug delivery, and the kit contains instructions describing oral administration of the dosage forms in a manner effective to treat a biofilm-associated infection.

The following examples are intended to illustrate but not limit the invention.

### EXAMPLES

### Example 1

### Efficacy of Gallium Maltolate in Pseudomonas aeruginosa Biofilm-Associated Urinary Tract Infections (UTI) in Mice

Catheter-associated UTI with Pseudomonas aeruginosa Xen5 (10⁶ CFU/catheter) in CF-1 female mice was established as described by Kadurugamuwa et al. (2005) Infection and Immunity 73(7):3878-87). Gallium maltolate (GaM) was assessed for efficacy against urinary tract infection (UTI) by P. aeruginosa, alone or in combination with ciprofloxacin (cipro) using bioluminescent engineered P. aeruginosa Xen5 in a CF-1 female mice UTI model that allows real time monitoring of infection with the Xenogen IVIS® Imaging System.

Catheter-associated UTI with P. aeruginosa (10⁶ colony forming units (CFU)/catheter) were established in mice and treated 2 days after infection for 4 consecutive days with GaM alone; or cipro alone; or the combination of GaM and cipro. A second cycle of treatment was started on days 9-11 and the study terminated on day 21. Controls included saline-treated uninfected and infected animals.

Ciprofloxacin US Pharmacopeia, Rockville, Md.) and gallium maltolate were administered by oral gavage. Ciprofloxacin was given in 0.2 mL of water and gallium maltolate in 0.2 mL water containing 1% carboxylmethyl cellulose. A second group of animals served as an untreated infection control group by being implanted with infected catheters and treated with saline. In another control group, animals were implanted with sterile catheters and served as a negative control.

Treatment of animals with catheter-associated infection commenced 2 days after bacterial challenge as indicated in Table 1.

TABLE 1
Summary of Animal Groups and Treatments

| | | | | Treatment |
|---|---|---|---|---|
| Group | Compound | No. of Mice | Dose | Frequency |
| 1 | Gallium | 4 | 30 mg/kg q.d. | Day 2-5 and 9-11 |
| | maltolate | | | |
| 2 | Gallium | 4 | 100 mg/kg q.d. | Day 2-5 and 9-11 |
| | maltolate | | | |
| 3 | Gallium | 4 | 300 mg/kg q.d. | Day 2-5 and 9-11 |
| | maltolate | | | |
| 4 | Ciprofloxacin | 8 | 30 mg/kg b.i.d. | Day 2-5 and 9-11 |
| 5 | Gallium | 4 | Gallium maltolate 30 | Day 2-5 and 9-11 |
| | maltolate + | | mg/kg q.d. + | |
| | Ciprofloxacin | | Ciprofloxacin 30 mg/kg | |
| | | | b.i.d | |
| 6 | Gallium | 4 | Gallium maltolate 100 | Day 2-5 and 9-11 |
| | maltolate + | | mg/kg q.d. + | |
| | Ciprofloxacin | | Ciprofloxacin 30 mg/kg | |
| | | | b.i.d | |
| 7 | Gallium | 4 | Gallium maltolate 300 | Day 2-5 and 9-11 |
| | maltolate + | | mg/kg q.d. + | |
| | Ciprofloxacin | | Ciprofloxacin 30 mg/kg | |
| | | | b.i.d | |
| 8 | Infected control | 12 | Saline q.d. | Day 2-5 and 9-11 |
| 9 | Uninfected | 6 | Saline q.d. | Day 2-5 and 9-11 |
| | control | | | |

All agents were given orally.
At end of experiment, one catheter from Groups 1, 2, 4, 6 and 8 were sent for EM analysis.

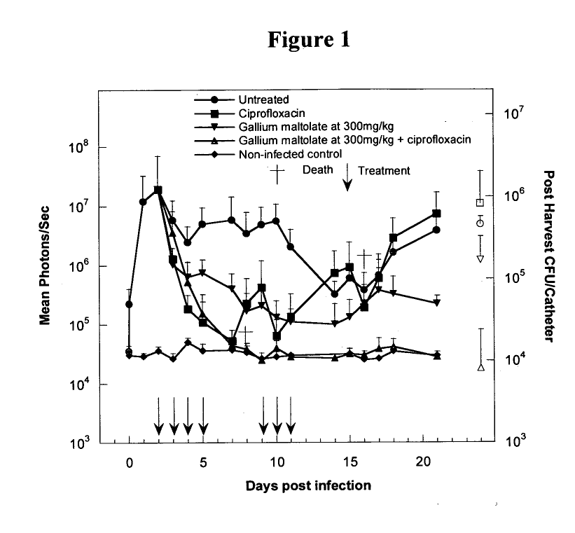

Therapeutic agents were readministered for 3 consecutive days, after 4 days of cessation of the initial 4-day therapy. Animals treated with the highest dose of gallium maltolate tested are shown in FIG. 1.

The bioluminescent signal recorded in the bladder of mice following the placement of a pre-colonized catheters, reached approximately 10⁷ Photons/Sec one day after implantation (FIG. 1). Compared to the untreated control groups, the ciprofloxacin treated animals showed a rapid decline in bioluminescent signal to almost nearly to undetectable levels after four days of treatments. However, the intensity of the bioluminescent signal began to increase approximately two days after discontinuing ciprofloxacin treatment, indicating a reestablishment of the infection upon cessation of antibiotic therapy. Interestingly, re-administration of antibiotic for three consecutive days, following two days of cessation of initial therapy, resulted in a repeat decline in the bioluminescence signal, as shown previously (Kadurugamuwa et al (2005) Infection and Immunity 73(7): 3878-87). However, soon after discontinuation of this bout of ciprofloxacin treatment the infection re-established once again and approximately reached untreated levels, suggesting significant bacterial regrowth in this cohort.

A reduction in bioluminescence following treatment with 300 mg/kg of gallium maltolate was also observed. Although the signal intensity never reached that of the non-infected group, the signal remained at a low level, approximately 10⁵ photons/catheter, until the termination of the experiment on day 21. Unlike the ciprofloxacin-treated group, the low level signal never increased significantly even after the termination of gallium maltolate administration, suggesting that recurrence of high levels of infection did not occur in this group of animals. Compared to the untreated control group, gallium maltolate dosed at 30 and 100 mg/kg also showed antibacterial growth activity (Table 2).

TABLE 2
Monitoring of P. aeruginosa Xen 5 growth during treatment with gallium
maltolate and ciprofloxacin

| | Percent bacterial growth in animals | | | | | |
|---|---|---|---|---|---|---|
| | compared to untreated control | | | | | |
| | Day | | | | | |
| Group | 2 | 5 | 8 | 10 | 15 | 21 |
| Untreated | 100% | 100% | 100% | 100% | 100% | 100% |
| Ciprofloxacin 30 mg/kg | 100% | 3% | 9% | 2% | 138% | 272% |
| Gallium maltolate 30 mg/kg | 100% | 60% | 10% | 13% | 54% | 22% |
| Gallium maltolate 100 mg/kg | 100% | 33% | 12% | 2% | 11% | 1% |
| Gallium maltolate 300 mg/kg | 100% | 19% | 7% | 4% | 20% | 8% |
| Gallium maltolate 30 mg/kg + | 100% | 3% | 10% | 5% | 11% | 6% |
| ciprofloxacin | | | | | | |
| Gallium maltolate 100 mg/kg + | 100% | 11% | 8% | 5% | 55% | 14% |
| ciprofloxacin | | | | | | |
| Gallium maltolate 300 mg/kg + | 100% | 4% | 1% | 1% | 5% | 1% |
| ciprofloxacin | | | | | | |

Comparison of antibacterial efficacy of gallium maltolate doses alone or in combination with the antibiotic ciprofloxacin. The results are presented as the percent growth of drug-treated bacteria compared to the bacterial growth of the untreated control group. The lower the percentage of growth for the drug-treated groups, the more active was the treatment against the infection.

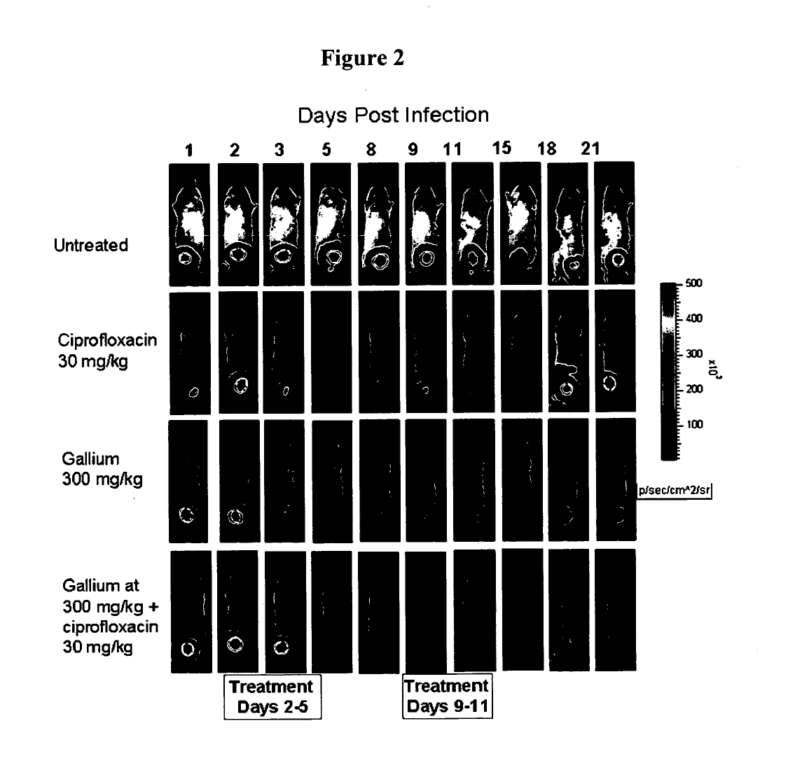

Interestingly, when infected animals were treated with the highest dose (300 mg/kg) of gallium maltolate and ciprofloxacin, a rapid decrease in the bioluminescent signal was observed (FIGS. 1 and 2, and Table 2). The combined treatment resulted in a reduction in bioluminescence to undetectable levels after four days of treatments. This dose of gallium maltolate did not produce adverse effects in orally fed mice suggesting the tolerability of this drug. Importantly, reestablishment of infection as seen in the group given ciprofloxacin alone was not observed when the antibiotic was given in combination with gallium maltolate, demonstrating synergy between gallium maltolate and ciprofloxacin in eradicating chronic infection.

TABLE 3
Mortality and rate of spread into the kidney in mice infected
in the bladder with P. aeruginosa Xen5

| | No. of animals with a | |
|---|---|---|
| | kidney signal at day | |
| Group | 21/Total no. of animals | % Mortality |
| Untreated | 4/7 | 41.7% |
| Ciprofloxacin 30 mg/kg | 1/7 | 12.5% |
| Gallium maltolate 30 | 0/3 | 25.0% |
| mg/kg | | |
| Gallium maltolate 100 | 0/3 | 25.0% |
| mg/kg | | |
| Gallium maltolate 300 | 0/4 | 0.0% |
| mg/kg | | |
| Gallium maltolate 30 | 0/4 | 0.0% |
| mg/kg + | | |
| Ciprofloxacin | | |
| Gallium maltolate 100 | 0/4 | 0.0% |
| mg/kg + | | |
| Ciprofloxacin | | |
| Gallium 300 mg/kg + | 0/4 | 0.0% |
| Ciprofloxacin | | |

Remarkably, animals given gallium maltolate alone and in combination with ciprofloxacin were free of kidney infections at the termination of the study, at all doses tested (Table 3). This may indicate an additional role for gallium in suppressing the spread of pathogen from the primary site of infection.

The bioluminescent signal in the untreated infected group remained stable initially but declined approximately by approximately half a log after ten days into the infection. This reduction in signal intensity is due to the death of infected animals with a strong signal. However, the signal intensity returned a few days later as the infection increased in severity in the surviving animals (FIG. 1).

TABLE 4
Bacterial counts in urine after infection with P. aeruginosa Xen 5

| | No. of days | Range of | |
|---|---|---|---|
| | after | bacterial counts | |
| Group | infection | in urine (CFU/ml) | Geometric Mean |
| Untreated | 7 | 3.0 × 10⁵-6.0 × 10⁵ | 9.0 × 10⁵ |
| | 11 | 1.4 × 10⁵-2.4 × 10⁷ | 2.9 × 10⁶ |
| | 14 | 6.0 × 10⁴-5.2 × 10⁵ | 2.1 × 10⁵ |
| | 16* | 4.0 × 10⁴-1.6 × 10⁶ | 2.5 × 10⁵ |
| | 17 | 4.0 × 10⁵-1.6 × 10⁶ | 7.9 × 10⁵ |
| | 21 | 1.2 × 10⁵-2.4 × 10⁶ | 9.1 × 10⁵ |
| Ciprofloxacin | 7 | 2.0 × 10²-7.0 × 10⁵ | 3.3 × 10³ |
| | 11 | 2.0 × 10² | 2.0 × 10² |
| | 14 | 2.0 × 10²-1.2 × 10⁷ | 1.1 × 10⁵ |
| | 16 | 2.0 × 10²-6.0 × 10⁵ | 3.2 × 10⁴ |
| | 17 | 2.0 × 10²-8.0 × 10⁶ | 1.2 × 10⁵ |
| | 21 | 2.0 × 10²-3.6 × 10⁷ | 2.2 × 10⁵ |
| Gallium maltolate | 7 | N/A | N/A |
| 30 mg/kg | 11 | 1.2 × 10⁵-1.8 × 10⁵ | 1.5 × 10⁵ |
| | 14 | N/A | N/A |
| | 16 | 1.2 × 10⁵-6.0 × 10⁵ | 2.4 × 10⁵ |
| | 17 | 4.0 × 10⁴-6.0 × 10⁵ | 1.6 × 10⁵ |
| | 21 | 1.2 × 10⁴-2.0 × 10⁶ | 1.6 × 10⁵ |
| Gallium maltolate | 7 | N/A | N/A |
| 100 mg/kg | 11 | 2.0 × 10²-6.0 × 10⁶ | 4.6 × 10⁴ |
| | 14 | N/A | N/A |
| | 16 | 2.0 × 10²-3.0 × 10⁶ | 1.1 × 10⁴ |
| | 17 | 2.0 × 10²-2.0 × 10⁶ | 2.9 × 10⁴ |
| | 21 | 2.0 × 10²-1.4 × 10⁶ | 8.2 × 10³ |
| Gallium maltolate | 7 | 1.3 × 10⁵-1.5 × 10⁶ | 3.0 × 10⁵ |
| 300 mg/kg | 11 | 2.2 × 10⁴-6.2 × 10⁵ | 1.1 × 10⁵ |
| | 14 | 1.2 × 10⁵-6.0 × 10⁵ | 2.3 × 10⁵ |
| | 16 | 3.2 × 10⁵-8 × 10⁵ | 5.8 × 10⁵ |
| | 17 | 4.0 × 10⁴-1.6 × 10⁶ | 3.2 × 10⁵ |
| | 21 | 1.6 × 10⁵-5.6 × 10⁶ | 3.1 × 10⁵ |
| Gallium maltolate | 7 | N/A | N/A |
| 30 mg/kg + | 11 | 2.0 × 10² | 2.0 × 10² |
| Ciprofloxacin | 14 | N/A | N/A |
| | 16 | 2.0 × 10²-4.0 × 10⁵ | 2.5 × 10³ |
| | 17 | 2.0 × 10²-2.0 × 10⁶ | 4.3 × 10³ |
| | 21 | 2.0 × 10²-2.8 × 10⁶ | 4.8 × 10³ |
| Gallium maltolate | 7 | N/A | N/A |
| 100 mg/kg + | 11 | 2.0 × 10² | 2.0 × 10² |
| Ciprofloxacin | 14 | N/A | N/A |
| | 16 | 2.0 × 10²-6.0 × 10⁶ | 6.2 × 10³ |
| | 17 | 2.0 × 10²-1.0 × 10⁶ | 3.4 × 10³ |
| | 21 | 2.0 × 10² | 2.0 × 10² |
| Gallium maltolate | 7* | 2.0 × 10² | 2.0 × 10² |
| 300 mg/kg + | 11 | 2.0 × 10² | 2.0 × 10² |
| Ciprofloxacin | 14 | 2.0 × 10² | 2.0 × 10² |
| | 16* | 2.0 × 10²-1.0 × 10⁴ | 1.4 × 10³ |
| | 17 | 2.0 × 10²-4.0 × 10³ | 5.4 × 10² |
| | 21 | 2.0 × 10²-3.1 × 10⁴ | 7.2 × 10² |

N/A: No urine CFU data available for that day
*Number of urine samples less than 3 due to unsuccessful sampling.
The lower limit of detection of bacteria by CFU method in urine < 10² CFU/ml

Pharmacokinetics

Adult, female CF-1 mice (initial weight range 32-39 gram) were treated with 30 and 300 mg/kg gallium maltolate by oral gavage q.d. for 4 consecutive days. Both doses were administered in the same volume of drug solution, i.e., 480 μL of 2.5 and 25 mg/mL gallium maltolate in 1% carboxyl methylcellulose. Blood was drawn at the following time points after the final dose administration: 0, 0.5, 1 3, 6, 9, 12, 24, 33 and 48 hours. Blood drawn at time point ‘0’ was taken immediately after dosing. Blood samples were collected within ±5% of the scheduled time point. Whole blood (500 to 1000 μL) was drawn from a single mouse by cardiac puncture, and four mice were sacrificed per time point for each dosing group. After blood collection, the specimens were held at room temperature to allow for clot formation followed by centrifugation to obtain serum. The concentration of gallium in 100 μL serum was determined by Inductively Coupled Plasma—Mass Spectrometry (ICP-MS).

After a weighted linear regression analysis calibration curve had been established and validated by quality control analysis, serum gallium concentrations in the samples were measured and the results plotted.

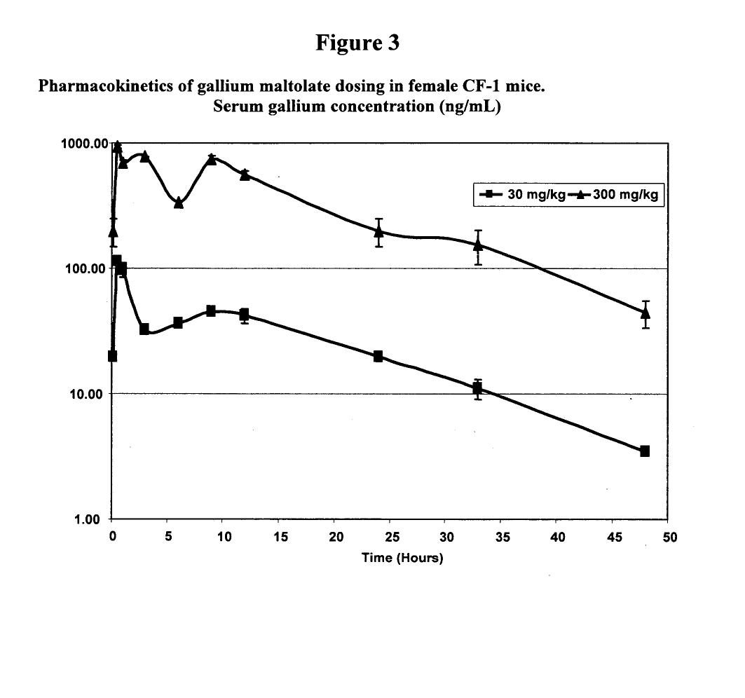

At both doses, serum gallium concentrations peaked within half hour of dosing and gradually declined over the 48 h assessment period with an initial half-life of elimination of 8-12 h. The results of the pharmacokinetic analysis are shown in FIG. 3.

Scanning Electron Microscopy Analysis of Longitudinal Sections of Explanted Catheters Bearing Pseudomonas aeruginosa Biofilms

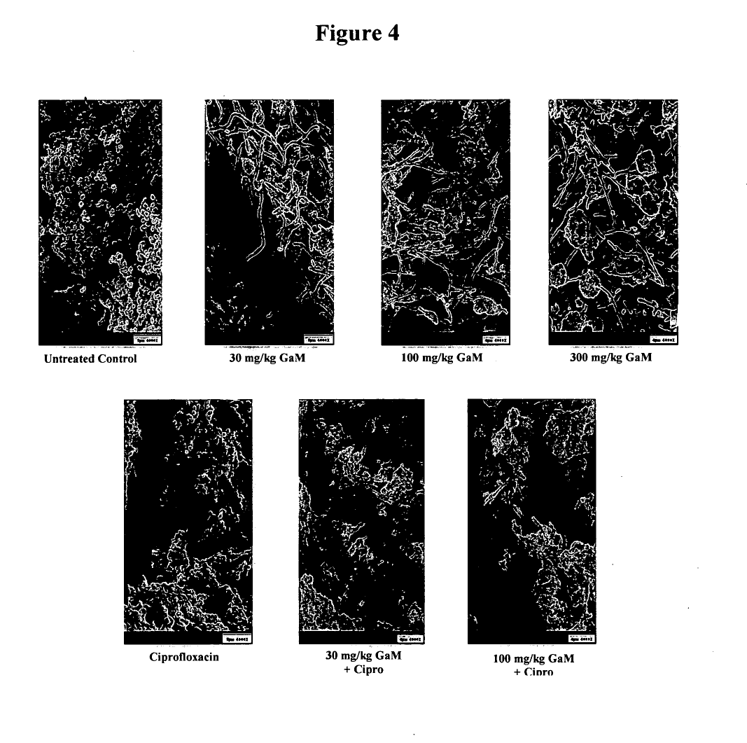

On day 21, 10 days after final dosing, one catheter from the following groups: Untreated control, 30 mg/kg GaM, 100 mg/kg GaM, 300 mg/kg GaM, 30 mg/kg Cipro, 30 mg/kg GaM plus 30 mg/kg Cipro, and 100 mg/kg GaM plus 30 mg/kg Cipro; were washed with PBS and placed in 2.5% glutaraldehyde fixative overnight at room temperature and then sent for scanning electron microscopy (SEM) analysis. The results are shown in FIG. 4.

Untreated bacteria appear as short rods embedded in a polymeric matrix in control catheter cross-sections. Unexpectedly, the morphological appearance of bacteria in catheters from gallium maltolate-treated groups appears filament-like, even at the lowest concentration tested. This striking alteration of the cellular morphology of the bacterial biofilm architecture and the associated reduction of the extracellular polymeric substance within the biofilm appears to correlate with treatment with increasing concentrations of gallium maltolate.

Treatment with ciprofloxacin did not appear to alter the short rod-like morphology of the bacteria. The bacterial cells appear tightly embedded in a polymeric matrix as seen with the untreated control group.

Combination treatment with gallium maltolate and ciprofloxacin resulted in elongated bacterial rods and filament-like bacteria. The tightly packed biofilm matrix seen in untreated or ciprofloxacin-treated groups appears less dense when ciprofloxacin is combined with gallium maltolate.

It is to be noted that the effects of gallium on bacterial morphology and biofilm architecture in these scanning electron micrographs were observed 10 days after final treatment with gallium maltolate, indicating that these novel effects persisted well after serum gallium was washed out.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes and to the same extent as if each individual publication, patent, or patent application were specifically and individually indicated to be so incorporated by reference.

We claim:

1. A method for treating a biofilm in an individual in need thereof, comprising administering a therapeutically effective amount of a gallium-containing composition to the individual, wherein said biofilm is at least at one site selected from the group consisting of the bladder, the kidney, the heart, the middle ear, the sinuses, the skin, a joint, subcutaneous tissue, soft tissue, vascular tissue, and the eye.

2. A method according to claim 1, wherein the biofilm is associated with a biofilm-associated urinary tract infection.

3. A method according to claim 1, wherein spread of the biofilm-associated infection to another site in the individual is inhibited.

4. A method according to claim 1, wherein the biofilm comprises at least one Gram-negative bacterial species.

5. A method according to claim 4, wherein the biofilm comprises Pseudomonas aeruginosa, Branhamella, Campylobacteria, Escherichia coli, Enterobacteria, Pasteurella, Proteus, Klebsiella, Neisseria, Salmonella, Shigella, or Serratia.

6. A method according to claim 1, wherein the biofilm comprises at least one Gram-positive bacterial species.

7. A method according to claim 6, wherein the biofilm comprises Bacillus, Corynebacteria, Clostridium, Enterococcus, Listeria, Staphylococcus, or Streptococcus.

8. A method according to claim 1, further comprising administering an antibiotic substance to the individual.

9. A method according to claim 8, wherein said antibiotic substance is selected from the group consisting of ciproflaxin, ampicillin, azithromycin, cephalosporin, doxycycline, gentamycin, levofloxacin, norfloxacin, ofloxacin, tetracycline, tobramycin, vancomycin, amikacin, deftazidime, cefepime, trimethoprim/sulfamethoxazole, piperacillin/tazobactam, aztreanam, meropenem, colistin, and chloramphenicol.

10. A method according to claim 8, wherein said gallium-containing composition and said antibiotic substance act synergistically to treat said biofilm.

11. A method according to claim 8, wherein said gallium-containing composition and said antibiotic substance act additively to treat said biofilm.

12. A method according to claim 1, wherein said gallium-containing composition comprises a coordination complex in the form of a neutral 3:1 (hydroxypyrone:gallium) complex in which each hydroxypyrone molecule is either unsubstituted or substituted with one, two, or three C₁-C₆ alkyl substituents.

13. A method according to claim 12, wherein each hydroxypyrone molecule is selected from the group consisting of 3-hydroxy-4-pyrone, 3-hydroyx-2-methyl-4-pyrone, 3-hydroxy-2-ethyl-4-pyrone, and 3-hydroxy-6-methyl-4-pyrone.

14. A method according to claim 13, wherein each hydroxypyrone molecule is 3-hydroxy-2-methyl-4-pyrone.

15. A method according to claim 1, wherein the gallium-containing composition is administered parenterally.

16. A method according to claim 1, wherein the gallium-containing composition is administered orally.

17. A method according to claim 1, wherein the gallium-containing composition is administered locally or topically.

18. A method according to claim 17, wherein the gallium-containing composition is administered locally to the eye as ophthalmic eye drops.

19. A method according to claim 1, wherein the biofilm is associated with chronic bacterial vaginosis.

20. A method according to claim 1, wherein the biofilm is associated with bacterial keratitis.

21. A method according to claim 8, wherein the gallium-containing composition and the antibiotic substance are administered simultaneously.

22. A method according to claim 8, wherein the gallium-containing composition and the antibiotic substance are administered sequentially.

23. A method for treating an orally-associated biofilm in an individual, comprising contacting an oral surface with a therapeutically effective amount of a gallium-containing composition.

24. A method according to claim 23, said method comprising preventing formation of a biofilm in said individual by administration of a prophylactically effective amount of said gallium-containing composition.

25. A method according to claim 24, wherein the orally-associated biofilm is dental plaque located on a tooth.

26. A method according to claim 24, wherein the gallium-containing composition is formulated as a dentrifice.

27. A method according to claim 26, wherein said dentrifice is a toothpasate composition.

28. A method according to claim 23, wherein said biofilm is located on the tongue, oral mucosa, or gum.

29. A method according to claim 23, wherein the gallium-containing composition is formulated as a mouthwash, a chewing gum, a paint, a foam, a gel, or a varnish.

30. A method according to claim 23, wherein said gallium-containing composition comprises a coordination complex in the form of a neutral 3:1 (hydroxypyrone:gallium) complex in which each hydroxypyrone molecule is either unsubstituted or substituted with one, two, or three C₁-C₆ alkyl substituents.

31. A method according to claim 30, wherein each hydroxypyrone molecule is selected from the group consisting of 3-hydroxy-4-pyrone, 3-hydroyx-2-methyl-4-pyrone, 3-hydroxy-2-ethyl-4-pyrone, and 3-hydroxy-6-methyl-4-pyrone.

32. A method according to claim 31, wherein each hydroxypyrone molecule is 3-hydroxy-2-methyl-4-pyrone.

33. A method according to claim 23, wherein spread of the biofilm to another site in the individual is inhibited.

34. A gallium-containing composition formulated for topical oral administration, wherein said composition is formulated as a dentrifice, a mouthwash, a chewing gum, a paint, a foam, a gel, or a varnish.

35. A gallium-containing composition formulated as an ophthalmic solution for topical administration to the eye.

36. A kit comprising a composition according to claim 34, and instructions for use in a method for treating or preventing an orally-associated biofilm.

37. A kit for use in the method of claim 1, comprising a gallium-containing composition and instructions for use in a method for treating a biofilm.

38. A kit according to claim 37, further comprising an antibiotic substance.
